(19) United States
(12) Patent Application Publication
(10) Pub. No.: US 2011/0061654 A1
(43) Pub. Date: Mar. 17, 2011

(54) OXYGEN MASK WHICH FACILITATES A PATEINT TO BREATH OXYGEN

(76) Inventor: Po-Yu KUO, Panchiao Taipei (TW)

(21) Appl. No.: 12/559,513

(22) Filed: Sep. 15, 2009

(51) Int. Cl. A61M 16/06

(52) U.S. Cl. 128/204.18

(57) ABSTRACT

The present invention discloses a structural improvement of an oxygen mask which covers a nose without covering a mouth, wherein a respiration tube faces upward, an air inlet tube is located at a center of an exterior side of the respiration tube and an inner tube of the air inlet tube is extended into the respiration tube. A patient breathes in oxygen from the air inlet tube and breathes out from a tube opening of an exhale tube at an upper end, so that he or she can inhale more oxygen.

### BACKGROUND OF THE INVENTION

a) Field of the Invention

The present invention relates to an oxygen mask which facilitates a patient to breath oxygen and more particularly to an oxygen mask which covers a nose without covering a mouth, so that more oxygen can be supplied when a patient is inhaling oxygen.

b) Description of the Prior Art

Referring to FIG. 1, as covering a mouth A and a nose B of a patient simultaneously, a conventional mask 100 is provided with a larger area and an imperfect bonding ability; besides, a metal piece 101 must be used to clip a nose bridge. On the other hand, as the mouth A is covered by the mask 100, it is not convenient for treatment and as a respiration tube 102 faces downward, it is even inconvenient for the physical examination. In addition, the aforementioned respiration tube 102 is provided with two air inlets 103 which supply air to the patient simultaneously when the patient inhales oxygen from the respiration tube 102. This oxygen inhaling method is not very helpful to the patient to inhale more oxygen.

### SUMMARY OF THE INVENTION

Accordingly, the primary object of the present invention to provide an oxygen mask which facilitates a patient to breath oxygen, which covers a nose without covering a mouth, wherein a respiration tube faces upward, an air inlet tube is located at a center of an exterior side of the respiration tube and an inner tube of the air inlet tube is extended into the respiration tube. A patient breathes in oxygen from the air inlet tube and breathes out from a tube opening of an exhale tube at an upper end, so that he or she can inhale more oxygen.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a schematic view illustrating actions of a prior art.

FIG. 2 shows a schematic view of the present invention.

FIG. 3 shows an exploded view of the present invention.

FIG. 4 shows a plan view illustrating actions of the present invention.

FIG. 5 shows a schematic view illustrating actions of a respiration tube of the present invention.

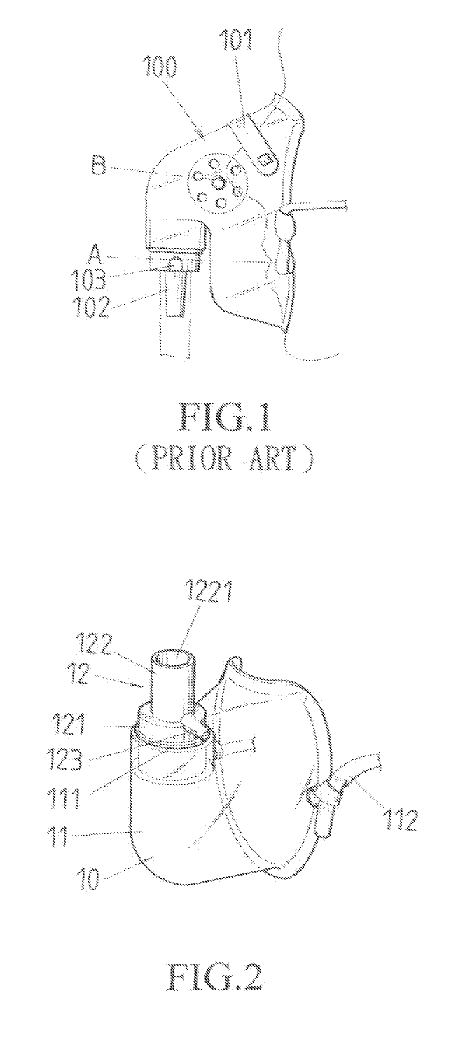

### DETAILED DESCRIPTION OF T HE PREFERRED EMBODIMENTS

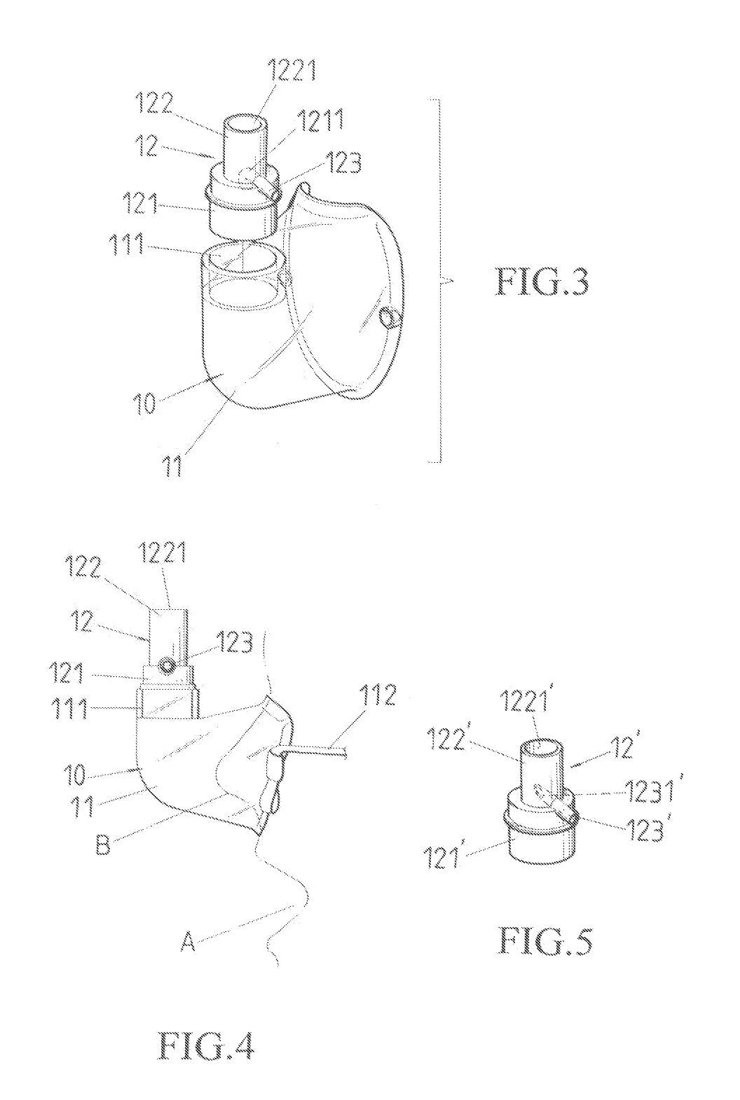

Referring to FIG. 2 to FIG. 4, an oxygen mask 10 of the present invention is small in size and only covers a nose B of a patient without covering a mouth A, the oxygen mask 10 comprises a mask 11 and a respiration tube 12, openings at two sides of the mask 10 are tied with straps 112 to sheath the mask 10 at a patient's head, a top part at a front side is provided with a latch slot 111 to latch a respiration tube 12. As the respiration tube 12 faces upward, a piping of the respiration tube 12 will not wind around a chest, thereby facilitating a physical examination.

The present invention is primarily characterized in that:

A transversal air inlet tube 123 is provided at a center of an exterior side of the respiration tube 12 and is extended to an inner tube 1231 inside the respiration tube 12 to be bended in an L-shape. An exhale tube 122 is located at an upper end of the respiration tube 12. When the patient inhales, he or she breathes in oxygen from the air inlet tube 123 of the respiration tube 12 and when the patient exhales, he or she breathes out from a tube opening 1221 of the exhale tube 122. Through repeated tests by a hospital, this oxygen inhaling method is the best to facilitate the patient breathing in oxygen and can supply more oxygen to breath in than an ordinary respiration tube.

Conclusive from the above, the present invention provides an oxygen mask which facilitates a patient to breath oxygen, which covers the nose without covering the mouth, wherein a respiration tube faces upward, an air inlet tube is located at a center of an exterior side of the respiration tube and an inner tube of the air inlet tube is extended into the respiration tube. A patient breathes in oxygen from the air inlet tube and breathes out from a tube opening of an exhale tube at an upper end, so that he or she can inhale more oxygen.

What is claim is:

1. An oxygen mask which facilitates a patient to breath oxygen comprising a mask and a respiration tube, wherein the mask covers a nose without covering a mouth, a top part at a front side of the mask is provided with a latch slot to latch a respiration tube, the respiration tube is connected with the oxygen mask with a tube opening facing upward to facilitate a physical examination; the primary characteristic being that a respiration tube faces upward, an air inlet tube is located at a center of an exterior side of the respiration tube and an inner tube of the air inlet tube is extended into the respiration tube to be bended in an L-shape, an exhale tube is located at an upper end of the respiration tube; A patient breathes in oxygen from the air inlet tube and breathes out from a tube opening of an exhale tube at an upper end.

2. The oxygen mask which facilitates a patient to breath oxygen in accordance with claim 1, wherein a tube opening of an inner tube in the respiration tube into which the air inlet tube is extended is provided with a beveled surface.
